(19) United States
(12) Patent Application Publication
(10) Pub. No.: US 2014/0018595 A1
(43) Pub. Date: Jan. 16, 2014

(54) PROCESS FOR REMOVING HYDROGEN HALIDES IN A RECTIFYING COLUMN WITH A PARTIAL CONDENSER

(71) Applicant: BASF SE, Ludwigshafen (DE)

(72) Inventors: Katharina Spuhl, Forest (BE); Stefan Bitterlich, Dirmstein (DE); Lukas Schulz, Mannheim (DE); Michael Hübner, Lampertheim (DE)

(73) Assignee: BASF SE, Ludwigshafen (DE)

(21) Appl. No.: 13/937,373

(22) Filed: Jul. 9, 2013

Related U.S. Application Data
(60) Provisional application No. 61/670,132, filed on Jul. 11, 2012.

(51) Int. Cl. C07C 7/00

(52) U.S. Cl. CPC C07C 7/00
USPC 585/800

(57) ABSTRACT

The present invention relates to a process for treating an output from a chemical reaction, wherein the chemical reaction is performed in the presence of an ionic liquid. The chemical reaction is preferably an isomerization. In a rectifying column with a partial condenser, the hydrogen halide is drawn off from a mixture which originates from the chemical reaction and comprises at least one organic compound, preferably at least one hydrocarbon, and at least one hydrogen halide.

### CROSS REFERENCE TO RELATED APPLICATION

This patent application claims the benefit of pending U.S. patent application Ser. No. 61/670,132 filed on Jul. 11, 2012, incorporated in its entirety herein by reference.

### BACKGROUND OF THE INVENTION

The present invention relates to a process for treating an output from a chemical reaction, wherein the chemical reaction is performed in the presence of an ionic liquid. The chemical reaction is preferably a hydrocarbon conversion, especially an isomerization. In a rectifying column with a partial condenser, the hydrogen halide is drawn off from a mixture which originates from the chemical reaction and comprises at least one organic compound, preferably at least one hydrocarbon, and at least one hydrogen halide.

Ionic liquids can be used in chemical reactions of organic compounds, for example in hydrocarbon conversion processes; they are especially suitable as catalysts for the isomerization of hydrocarbons. A corresponding use of an ionic liquid is disclosed, for example, in WO 2011/069929, where a specific selection of ionic liquids is used in the presence of an olefin for isomerization of saturated hydrocarbons, more particularly for isomerization of methylcyclopentane (MCP) to cyclohexane. A similar process is described in WO 2011/069957, but the isomerization therein is not effected in the presence of an olefin, but with a copper(II) compound.

In addition to the ionic liquid, in chemical reactions, especially in isomerization processes, it is also possible to use hydrogen halides, preferably as cocatalysts. Frequently, the hydrogen halides are used in gaseous form. In order to be able to better utilize the cocatalytic effect of the hydrogen halides, a partial pressure of 1-10 bar of hydrogen halide, especially of hydrogen chloride, is generally established over the reaction mixture in which the chemical reaction, especially an isomerization, is performed. However, a certain portion of the hydrogen halide used is dissolved in the organic compounds, especially in the hydrocarbons, and consequently discharged from the reaction. This proportion of hydrogen halide dissolved in the hydrocarbons has to be removed again from the hydrocarbons after the chemical reaction, particularly due to the corrosive properties of the hydrogen halide, and this removal is in practice frequently associated with problems.

US-A 2011/0155632 discloses a processes for preparing products with a low hydrogen halide content, wherein the content of hydrogen halides is reduced in at least two separation steps, by stripping or distillation from a mixture which originates from a reactor and comprises an ionic liquid as a catalyst. In one embodiment of the process described in US-A 2011/0155632, the ionic liquid used as a catalyst is recycled into an alkylation reactor from a downstream phase separator, and hydrogen chloride is recycled from a first distillation column downstream of the phase separator and an isobutane-comprising stream from a second distillation column further downstream into the alkylation reactor. US-A 2011/0155632, however, does not disclose anywhere that a hydrogen halide, especially hydrogen chloride, can be removed effectively via a one-stage separation step using a rectifying column with a partial condenser from a product, for example from an alkylation product or an isomerization product. In contrast, in the execution variants described therein, the use of two separation steps, especially of two distillation columns, is absolutely necessary in order to obtain a low content of hydrogen halide in the reaction product. A similar disclosure to that in US-A 2011/0155632 is present in US-A 2011/0155640, but the process described therein relates to a hydrocarbon conversion.

The term “distillation” which is performed in a corresponding distillation apparatus (distillation column) is generally understood by the person skilled in the art to mean the following: a characteristic feature of distillation is that a portion of the liquid mixture to be separated is vaporized with supply of heat and is condensed after removal from the remaining liquid mixture. A mixture in which the lower-boiling mixture components with respect to the mixture to be separated are enriched is thus obtained. Typically, the term “distillation” includes distillation in a column, also called “rectification”. This involves conducting the vapor generated by distillation within the column, also called rectifying column, in countercurrent to a portion of the condensate thereof and bringing them into intensive contact by means of suitable internals, for example trays or structured packings. In this way, more volatile components are enriched in the top product and less volatile components in the bottom product of the rectifying column (see also Perry's Chemical Engineer's Handbook (8th Edition), by Don W. Green and Robert H. Perry, © 2008 McGraw-Hill, Section 13, “INTRODUCTION TO DISTILLATION OPERATIONS, General Principles” section, see FIG. 13-1 therein).

Thus, the person skilled in the art, with regard to the removal of the hydrogen halide from the hydrocarbons in the reaction output, is faced with the task of configuring this removal step with maximum selectivity, i.e. in such a way that, on the one hand, the mixture depleted of hydrogen halide resulting from this step is very low in hydrogen halide, in order to minimize the corrosiveness of the mixture or the complexity of any necessary further process steps for hydrogen halide removal, and that, on the other hand, the mixture enriched in hydrogen halide resulting from this step is significantly enriched with respect to hydrogen halide, in order to avoid a large recycle stream and the associated process technology disadvantages (increase in energy demands, apparatus size and pipelines).

A distillation, if it is performed as a rectification in a column, enables more selective removal of the HX than, for example, a one-stage evaporation. However, when using a distillation column (as shown, for example, in the above reference from Perry's), the problem is encountered that, especially in the case of hydrocarbons having at least 5 carbon atoms, an extremely large temperature difference between the top and bottom of the column occurs, this corresponding approximately to the boiling point difference between the hydrocarbon and HX at the given pressure. For example, in the case of X═Cl and C6 hydrocarbons, this is >200 K. This consequence of this is in turn that either the top temperature has to be well below the customary cooling water temperatures, which necessitates an additional cooling unit, and/or the bottom temperature is above the temperature of customary energy carriers (for example steam at 16 bar gauge), which entails additional cost and inconvenience for the provision of the necessary bottoms heating.

U.S. Pat. No. 3,271,467 discloses a process and a corresponding apparatus for maintaining the hydrogen halide concentration in a hydrocarbon conversion, wherein the catalyst used is a metal halide and the hydrogen halide is used as a promoter. Suitable metal halides are, for example, aluminum chloride, aluminum bromide, boron trifluoride or halides of zinc, tin, antimony or zirconium, but such compounds are not ionic liquids. The hydrocarbon conversion may, for example, be an isomerization of methylcyclopentane (MCP) to cyclohexane. In a (first) stripping apparatus, a stream rich in gaseous hydrogen halide is removed from the hydrocarbon-containing output from the hydrocarbon conversion and discharged from the arrangement. A second stream enriched in hydrogen halide is passed from the stripping apparatus into an absorption apparatus, in order to selectively remove the hydrogen halide present in this stream over a solid absorber therein. The hydrogen halide thus removed is removed again from the solid absorber and recycled into the system.

WO 2010/075038 discloses a process for reducing the content of organic halides in a reaction product which is formed as a result of a hydrocarbon conversion process in the presence of a halogen-comprising catalyst based on an acidic ionic liquid. The hydrocarbon conversion process is especially an alkylation; this process can optionally also be performed as an isomerization. The organic halides are removed from the reaction product by washing with an aqueous alkaline solution. The use of hydrogen halide as a cocatalyst of ionic liquids in hydrocarbon conversions such as isomerization processes and the associated removal of hydrogen halide from the isomerization product, however, is not disclosed in WO 2010/075038.

### BRIEF SUMMARY OF THE INVENTION

It is an object of the present invention to provide a novel process for removing hydrogen halide from a mixture which is obtained in a chemical reaction, especially in an isomerization, of at least one organic compound in the presence of an ionic liquid.

The object is achieved by a process for treating an output from a chemical reaction in the presence of an ionic liquid, the output comprising a mixture (G1) and mixture (G1) comprising at least one organic compound and at least one hydrogen halide (HX), which comprises feeding mixture (G1) into a rectifying column (R1) comprising at least one partial condenser and at least one reboiler, and removing from (R1) a mixture (G1b) comprising at least 50% by weight of the hydrogen halide (HX) present in mixture (G1), and a mixture (G2) comprising a reduced amount of hydrogen halide (HX) compared to mixture (G1).

### BRIEF DESCRIPTION OF THE SEVERAL VIEWS OF THE DRAWINGS

FIG. 1 illustrates the process according to the invention in a preferred embodiment.

FIG. 2 illustrates the process according to the invention in a further preferred embodiment.

FIG. 3 schematically shows Example 1.

FIG. 4 schematically shows comparative Example 2.

### DETAILED DESCRIPTION

By virtue of the process according to the invention, it is advantageously possible to remove hydrogen halide present/dissolved in the corresponding product (hydrocarbons) after a chemical reaction, preferably a hydrocarbon conversion, especially an isomerization, from this product again, especially from an isomerization product. An important advantage is considered to be that, more particularly, due to the use of a rectifying column equipped with at least one partial condenser and at least one reboiler, it is possible to achieve significant depletion, preferably selective removal, of hydrogen halide, especially of hydrogen chloride, with a moderate temperature difference between the top and bottom or the rectifying column (R1) used.

In the context of the present invention, partial condensation is understood to mean that the vapors obtained at the top of the column (mixture (G1b)) is not condensed completely, i.e. to an extent of 100%; instead, a certain proportion (generally at least 0.1% by weight) remains in the gaseous state, which is either utilized as an offgas stream or, as is preferred in the context of the present invention, passed back into the apparatus V1. A total condensation in which the full proportion of the vapors is condensed at the top and no proportion remains in gaseous form, in contrast, would produce an extreme temperature difference between top and bottom. In this case, an enormous level of cooling would be needed in the condenser, and this would not be possible with the standard coolants in the field of distillation (cooling water or air).

The advantage of a moderate temperature difference between the top and bottom of the rectifying column (R1) used is enhanced when the optional steps a) to c) detailed below are performed, i.e. the vapors obtained at the top of the column (mixture (G1b)) are condensed to an extent of at most 98% by weight, preferably at most 95% by weight, and the condensate obtained is recycled as reflux to the column to an extent of at least 90%, preferably at least 95%.

In addition, the uncondensed portion of the vapors (mixture (G1b)), which comprises preferably between 10 and 90% by weight, more preferably between 30 and 80% by weight, of HX, is removed and, optionally after intermediate compression, recycled into the reaction to an extent of at least 80%. Thus, a further advantage is considered to be that, due to the optional, at least partial recycling of the hydrogen halide, this can additionally be reused in the process.

The advantages (smaller temperature difference) are additionally apparent when the feed of mixture (G1) is at a point below the column reflux, particular preference being given to a feed point above the column bottom, such that the column has a stripping section and rectifying section. In this embodiment, the column is preferably operated in such a way that a temperature in the stripping section is regulated with the heat output introduced into the reboiler, for example in the form of steam. In a further preferred embodiment, the temperature in the partial condenser is likewise regulated, this being effected, for example, by adjusting the amount or temperature of the cooling medium or else of the liquid level in the condenser.

The above-described advantages of the process according to the invention become even more apparent when the feeding of mixture (G1) into the rectifying column (R1) is preceded by an upstream connection of a phase separation unit, especially a phase separator, to the rectifying column (R1).

The process according to the invention for treatment of an output from a chemical reaction in the presence of an ionic liquid is defined in detail hereinafter.

The expression “chemical reaction” or “chemical reaction process” is understood in the context of the present invention, in principle, to mean any chemical conversion or chemical reaction which is known to those skilled in the art and in which at least one organic compound, especially at least one hydrocarbon, is chemically converted, modified or altered in terms of its composition or structure in some other way.

The chemical reaction is preferably a hydrocarbon conversion, especially an isomerization. The hydrocarbon conversion is preferably selected from an alkylation, a polymerization, a dimerization, an oligomerization, an acylation, a metathesis, a polymerization or copolymerization, an isomerization, a carbonylation or combinations thereof. Alkylations, isomerizations, polymerizations etc. are known to those skilled in the art. More preferably in the context of the present invention, the hydrocarbon conversion is an isomerization.

In the context of the present invention, the chemical reaction proceeds in the presence of an ionic liquid. For example, mixtures of two or more ionic liquids may be used, preference being given to using one ionic liquid.

Suitable ionic liquids in the context of the present invention are in principle all ionic liquids known to those skilled in the art. An overview with regard to suitable ionic liquids can be found, for example, in WO 2011/069929. In the context of the present invention, preference is given to an acidic ionic liquid. The ionic liquid present in phase (A) is preferably an ionic liquid, especially an acidic ionic liquid, having the composition K1AlₙX₍₃ₙ₊₁₎ where K1 is a monovalent cation, X is halogen and 1<n<2.5. K1 is preferably an unsubstituted or at least partly alkylated ammonium ion or a heterocyclic (monovalent) cation, especially a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. X is preferably chlorine or bromine.

The ionic liquid, especially the acidic ionic liquid, more preferably comprises, as a cation, an at least partly alkylated ammonium ion or a heterocyclic cation and/or, as an anion, a chloroaluminate ion having the composition AlₙCl₍₃ₙ₊₁₎ where 1<n<2.5. The at least partly alkylated ammonium ion preferably comprises one, two or three alkyl radicals (each) having 1 to 10 carbon atoms. If two or three alkyl substituents are present with the corresponding ammonium ions, the respective chain length can be selected independently; preferably, all alkyl substituents have the same chain length. Particular preference is given to trialkylated ammonium ions having a chain length of 1 to 3 carbon atoms. The heterocyclic cation is preferably an imidazolium ion or a pyridinium ion.

The ionic liquid, especially the acidic ionic liquid, especially preferably comprises, as a cation, an at least partly alkylated ammonium ion and, as an anion, a chloroaluminate ion having the composition AlₙCl₍₃ₙ₊₁₎ where 1<n<2.5. Examples of such particularly preferred ionic liquids are trimethylammonium chloroaluminate and triethylammonium chloroaluminate.

The ionic liquid used in the context of the present invention is preferably used as a catalyst in the chemical reaction, preferably in the hydrocarbon conversion, especially as an isomerization catalyst. In addition, in the context of the present invention, the chemical reaction is also effected in the presence of a hydrogen halide (HX), preference being given to using the hydrogen halide (HX) as a cocatalyst. The hydrogen halide can be actively added to the chemical reaction and/or else it can be eliminated during the chemical reaction, for example from the ionic liquid.

The hydrogen halides (HX) used may in principle be any conceivable hydrogen halides, for example hydrogen fluoride (HF), hydrogen chloride (HCl), hydrogen bromide (HBr) or hydrogen iodide (HI). The hydrogen halides can optionally also be used as a mixture, but preference is given in the context of the present invention to using only one hydrogen halide. Preference is given to using the hydrogen halide whose halide moiety is also present in the above-described (acidic) ionic liquid (at least partly) in the corresponding anion. The hydrogen halide (HX) is preferably hydrogen chloride (HCl) or hydrogen bromide (HBr). The hydrogen halide (HX) is more preferably hydrogen chloride (HCl).

In principle, it is possible in the context of the present invention to use any organic compounds, provided that at least one of the organic compounds used can be subjected in the presence of the above-described ionic liquids to a chemical reaction, preferably to a hydrocarbon conversion, especially to an isomerization. On the basis of his or her specialist knowledge, the person skilled in the art knows which organic compounds can be subjected by means of ionic liquids to a chemical reaction, preferably to a hydrocarbon conversion, and more particularly which organic compounds are isomerizable. For example, it is possible to use mixtures of two or more organic compounds, but it is also possible to use only one organic compound. Thus, it is possible in the context of the present invention that, in a mixture comprising two or more organic compounds, only one of these organic compounds is subjected to a chemical reaction, preferably to a hydrocarbon conversion, especially isomerized. Optionally, such mixtures may also comprise compounds which are not themselves organic compounds but are miscible therewith.

Organic compounds are understood in the context of the present invention to mean all compounds formed completely or at least partially from hydrocarbons. The organic compounds may, as well as the hydrocarbon constituent, also comprise (individual) heteroatoms such as sulfur, nitrogen or oxygen and/or functional groups such as hydroxyl, carbonyl, amino, amido, halogen, oxo and the like. Examples thereof are heteroaromatics such as pyridine or imidazole, heterocycles such as piperidine or imidazolidine, acids such as acetic acid or higher fatty acids having, for example 18 carbon atoms, ethers such as diethyl ether, amines such as ethylenediamine or aniline, or amides such as carboxamides. Organic compounds in the context of the present invention are, however, not understood to mean polymers having a molecular weight of >200 g/mol, salts or ionic liquids, even if these compound classes may at least partly also have hydrocarbon constituents.

Hydrocarbons (as such) are understood in the context of the present invention to mean those compounds as a portion of the organic compounds which are formed exclusively from hydrocarbons, and thus do not comprise any heteroatoms or have them as substituents. Hydrocarbons may be linear, cyclic or branched aliphatics or aromatics, for example alkanes such as cyclohexane, n-hexane or isohexanes, alkenes such as ethylene or propylene, and aromatics such as benzene or toluene. The hydrocarbons in the context of the present invention are more preferably linear, cyclic and/or branched alkanes.

The hydrocarbon used in the chemical reaction, preferably in the hydrocarbon conversion, is preferably methylcyclopentane (MCP) or a mixture of methylcyclopentane (MCP) with at least one further hydrocarbon selected from cyclohexane, n-hexane, isohexanes, n-heptane, isoheptanes, methylcyclohexane or dimethylcyclopentanes.

More preferably, a mixture of methylcyclopentane (MCP) with at least one further hydrocarbon selected from cyclohexane, n-hexane, isohexanes, n-heptane, isoheptanes, methylcyclohexane or dimethylcyclopentanes is used, the proportion of branched hydrocarbons in the mixture being greater than 50% by weight (based on the sum of all hydrocarbons).

The chemical reaction can in principle be performed in all apparatuses known for such a purpose to the person skilled in the art. If the chemical reaction/the hydrocarbon conversion is an isomerization, the corresponding apparatus is preferably a stirred tank or a stirred tank cascade. A “stirred tank cascade” means that two or more, for example three or four, stirred tanks are connected in succession (in series).

As already explained above, due to the chemical reaction in the presence of an ionic liquid and of a hydrogen halide (HX), the chemical structure of at least one of the organic compounds used, especially of the hydrocarbons, is altered. The organic compounds, preferably hydrocarbons, obtained in the chemical reaction, especially in the hydrocarbon conversion, are present in a mixture (G1). In other words, this means that mixture (G1) comprises the product obtained in the chemical reaction/hydrocarbon conversion (hydrocarbons with or without a heteroatom) in full or at least in part, preferably in full. The mixture (G1) thus differs in terms of the composition and/or amount of the organic compounds, preferably hydrocarbons, present therein from the corresponding composition prior to the isomerization. Since the chemical reaction to be conducted in such chemical reactions, preferably in hydrocarbon conversions, especially in isomerization processes, frequently does not proceed to an extent of 100% (i.e. to completion), the product generally still also comprises the organic compound, preferably the hydrocarbon, with which the chemical reaction/hydrocarbon conversion has been conducted (in a smaller amount than before the reaction). If, for example, MCP is to be isomerized to cyclohexane, the isomerization product frequently comprises a mixture of cyclohexane and (in a smaller amount than before the reaction) MCP.

As well as the organic compounds, preferably the hydrocarbons, mixture (G1) preferably comprises at least one hydrogen halide (HX) and optionally further components. The hydrogen halide (HX) present in mixture (G1) is generally the same hydrogen halide as that used in the chemical reaction (preferably as a cocatalyst), because the chemical structure of the hydrogen halide is not normally altered by the chemical reaction, but there may be partial exchange of the anionic moiety of the hydrogen halide used with other halide ions present in the process. As a further component, mixture (G1) preferably comprises the above-described ionic liquid. If mixture (G1) additionally comprises an ionic liquid, mixture (G1) additionally comprises between 10 and 99% by weight, preferably between 50 and 95% by weight, of ionic liquid (the stated amounts are based on the total weight of organic compounds, preferably hydrocarbons, and hydrogen halide in mixture (G1)).

The organic compound, especially the hydrocarbon, present in mixture (G1) is preferably cyclohexane. The hydrocarbon present in mixture (G1) is more preferably cyclohexane or a mixture of cyclohexane with at least one further hydrocarbon selected from methylcyclopentane (MCP), n-hexane, isohexanes, n-heptane, isoheptane, methylacyclohexane or dimethylcyclopentanes.

The hydrocarbon present in mixture (G1) is especially preferably a mixture of cyclohexane, MCP and at least one further hydrocarbon. The further hydrocarbon is preferably selected from n-hexane, isohexanes, n-heptane, isoheptanes, methylcyclohexane or dimethylcyclopentanes. If the chemical reaction/hydrocarbon conversion performed is an isomerization, the proportion of branched open-chain hydrocarbons in mixture (G1) is preferably less than 10% by weight (based on the sum of all hydrocarbons present in mixture (G1)). Particular preference is given in the context of the present invention to isomerizing methylcyclopentane (MCP) to cyclohexane.

In a preferred embodiment of the present invention, mixture (G1) comprises i) as a hydrocarbon a mixture of cyclohexane with at least one further hydrocarbon selected from methylcyclopentane (MCP), n-hexane, isohexanes, n-heptane, isoheptanes, methylcyclohexane and dimethylcyclopentane, ii) hydrogen chloride (HCl) and iii) an acidic ionic liquid which has, as a cation, an at least partly alkylated ammonium ion and, as an anion, a chloroaluminate ion having the composition AlₙCl₍₃ₙ₊₁₎ where 1<n<2.5.

In a further preferred embodiment of the present invention, the hydrocarbons present in mixture (G1), to an extent of at least 80% by weight, have at least 5 carbon atoms per molecule. These hydrocarbons are preferably present to an extent of at least 95% by weight in mixture (G1); it is additionally preferred that these hydrocarbons have at least 6 carbon atoms per molecule.

Mixture (G1) is at first present in the apparatus in which the chemical reaction is performed. In the context of the process according to the invention, mixture (G1) is removed from this apparatus as the output. In other words, this means that the output comprises mixture (G1) and the output or mixture (G1), after it has left the apparatus for performance of the chemical reaction, is fed into the rectifying column (R1) which comprises a partial condenser and a reboiler.

In the context of the present invention, the term “rectification”, which is performed in a corresponding rectification apparatus (also called rectifying apparatus, rectifying column or rectification column), is understood to mean the following: In rectification, the vapor produced by distillation is conducted in countercurrent to a portion of the condensate thereof in a rectifying column. In this way, more volatile components are enriched in the top product and less volatile components in the bottom product of the rectifying column. Further information regarding the term “rectification”, but also regarding other terms/techniques such as distillation, vaporization, flashing or stripping, can be found in the following textbooks: Sattler, Thermische Trennverfahren [Thermal Separation Processes], VCH, 1988; Perry's Chemical Engineers' Handbook, 7th edition; R. H. Perry, D. W. Green, 1997, McGraw-Hill, Section 13.

The rectifying column (R1) used to perform the removal of the hydrogen halide (HX) from mixture (G1) may in principle be any rectifying column known for such a purpose to the person skilled in the art, provided that the rectifying column comprises a partial condenser and a reboiler. This means that (R1) is equipped with at least one partial condenser and at least one reboiler. Both the reboiler and the partial condenser may be integrated in (R1) as one apparatus, but this is not obligatory. The partial condenser is preferably outside the actual column section of the rectifying column.

In the context of the present invention, a partial condensation of the mixture (G1b) is performed in the partial condenser which forms part of the rectifying column (R1). This means that at least 0.1% by weight, preferably 0.5% by weight, more preferably 2% by weight and even more preferably 5% by weight of mixture (G1b)—based on the total amount thereof—is not condensed in the partial condenser but remains gaseous.

The rectifying column (R1) is preferably configured such that is has a stripping section and the rectifying section. In this embodiment, (R1) is preferably operated in such a way that the temperature in the stripping section is regulated with the heat output introduced into the reboiler of (R1). It is additionally preferred that heat output is introduced into the reboiler in the form of steam or steam condensate. It is additionally preferred that the temperature in the partial condenser is regulated via the amount and/or temperature of the cooling medium and/or the liquid level in the partial condenser.

In the rectifying column (R1), at least a portion of the hydrogen halide present in mixture (G1) is removed therefrom as mixture (G1b). Thus, a mixture (G1b) comprising at least 50% by weight of the hydrogen halide (HX) present in mixture (G1), and a mixture (G2) comprising a reduced amount of hydrogen halide (HX) compared to mixture (G1), are removed (from one another) or drawn off from rectifying column (R1).

Mixture (G1b) is drawn off from rectifying column (R1) partly or fully in gaseous form, more preferably fully in gaseous form. Mixture (G1b) preferably comprises at least 70%, more preferably at least 95%, especially preferably at least 99%, of the hydrogen halide supplied with (G1).

Mixture (G2) is at first defined in that it comprises an amount of at least one hydrogen halide (HX) reduced by the amount of hydrogen halide present in mixture (G1b) relative to mixture (G1). Preferably, mixture (G2) comprises an amount of at least one hydrogen halide (HX) reduced relative to mixture (G1) by at least 70%, more preferably at least 95%, especially preferably at least 99%.

In addition, mixture (G2) comprises at least one organic compound, preferably at least one hydrocarbon. In the context of the process according to the invention, it is preferable that mixture (G2) obtained from the rectifying column (R1), with regard to the composition and/or amount of the organic compounds present therein, especially of the hydrocarbons, corresponds completely or at least substantially to mixture (G1). The expression “corresponds substantially” shall be understood in this context to mean that at least 90% by weight, preferably at least 95% by weight, especially at least 98% by weight, of the amount of organic compounds, preferably of the hydrocarbons, present in mixture (G1) is also present in mixture (G2).

A preferred embodiment of the present invention comprises the following steps (a to c):
a) drawing off the gaseous mixture (G1b) from the rectifying section of the rectifying column (R1), mixture (G1b) comprising at least 50% of the amount of the hydrogen halide (HX) present in mixture (G1),
b) partially condensing gaseous mixture (G1b) drawn off in step a) in the partial condenser of (R1) to obtain at most 98% by weight, preferably at most 95% by weight, of the total amount of (G1b) as condensate (KD), and removing the uncondensed portion of the gaseous mixture (G1b) comprising 10 to 90% by weight of hydrogen halide (HX) from the partial condenser,
c) at least partially recycling the condensate (KD) obtained in step b) into the rectifying column (R1).

It is additionally preferable that mixture (G1) is fed into the rectifying column (R1) at a point below the feed of the condensate (KD) to (R1) and/or above the column bottom of (R1).

In a preferred embodiment of the present invention, mixture (G1b) drawn off from the rectifying column (R1) is recycled fully or partly into the apparatus in which the chemical reaction, preferably the hydrocarbon conversion, especially the isomerization, is performed. More particularly, in this embodiment, the hydrogen halide (HX) present in mixture (G1b) is recycled fully or partly into the corresponding apparatus. Especially preferably, the at least partial recycling of the mixture (G1b) drawn off from the rectifying column (R1) is effected in combination with the above-described three-stage embodiment according to steps a) to c). If complete recycling of mixture (G1b) is not effected, any excess amounts of mixture (G1b) can be discharged from the process according to the invention and (generally) discarded or sent to a further process step.

In a further embodiment of the present invention, it is preferable that mixture (G2) removed from the rectifying column (R1) is liquid when it leaves (R1) and is at most 150 K, preferably at most 100 K, hotter than the gaseous mixture (G1b) when it leaves the rectifying section of (R1).

If the chemical reaction/hydrocarbon conversion in the context of the present invention is an isomerization, the isomerization is preferably performed as follows. The performance of an isomerization of hydrocarbons in the presence of an ionic liquid as a catalyst and a hydrogen halide as a cocatalyst is known to those skilled in the art. The hydrocarbons and the ionic liquid in the isomerization preferably each form a separate phase, though portions of the ionic liquid may be present in the hydrocarbon phase and portions of the hydrocarbons in the ionic liquid phase. The hydrogen halide, especially hydrogen chloride, is introduced, preferably in gaseous form, into the apparatus for performance of the isomerization. The hydrogen halide may be present, at least in portions, in the two aforementioned liquid phases; the hydrogen halide preferably forms a separate, gaseous phase.

The isomerization is preferably performed at a temperature between 0° C. and 100° C., especially preferably at a temperature between 30° C. and 60° C. It is additionally preferred that the pressure in the isomerization is between 1 and 20 bar abs. (absolute), preferably between 2 and 10 bar abs.

The isomerization is preferably performed in the apparatus in such a way that two liquid phases and one gaseous phase are present in a stirred tank or a stirred tank cascade. The first liquid phase comprises the acidic ionic liquid to an extent of at least 90% by weight and the second liquid phase comprises the hydrocarbons to an extent of at least 90% by weight. The gas phase comprises at least one hydrogen halide, preferably hydrogen chloride, to an extent of at least 90% by weight. Optionally, a solid phase may also be present, this comprising components from which the ionic liquid is formed in solid form, for example AlCl₃. The pressure and composition of the gas phase are set here such that the partial pressure of the gaseous hydrogen halide, especially of HCl gas, in the gas phase is between 1 and 20 bar abs., preferably between 2 and 10 bar abs.

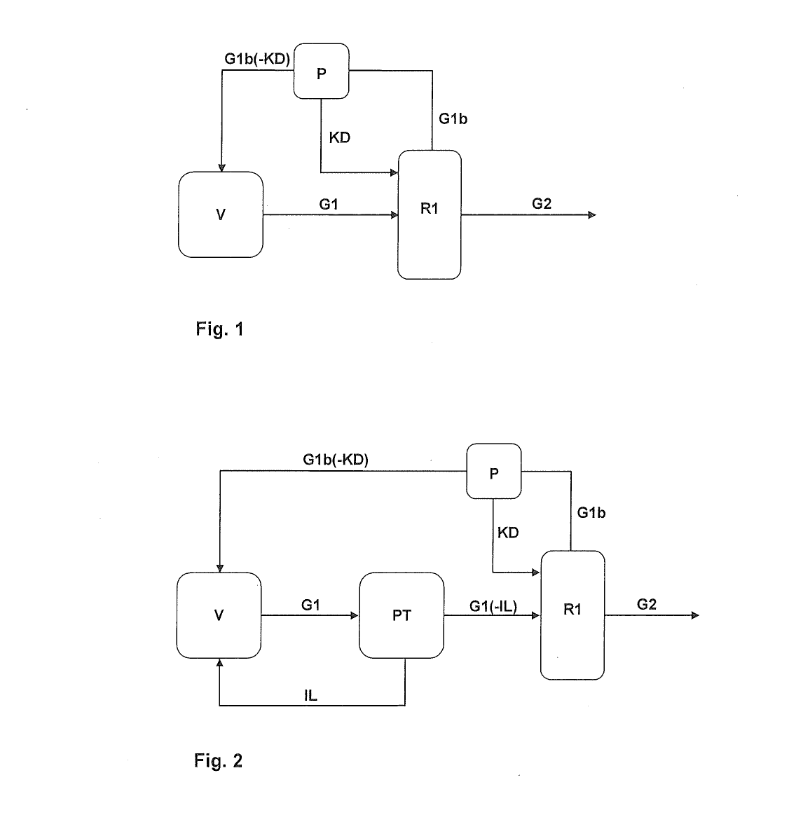

FIG. 1 once again illustrates the process according to the invention in a preferred embodiment. “V” represents the apparatus in which the chemical reaction, preferably the hydrocarbon conversion, especially an isomerization, is performed. This is preferably a stirred tank or a stirred tank cascade. In this embodiment, partial recycling of the mixture (G1b) removed from the rectifying column (R1), preferably in gaseous form, into the apparatus (V) is also performed. This recycling is effected in combination with the above-described additional steps a) to c), according to which a portion of the condensate (KD) obtained in the partial condenser is (likewise) recycled to R1. Mixture (G1) is preferably fed into (R1) at a point below the feed of (KD) to (R1). Optionally, in the embodiment of the present invention shown in FIG. 1, mixture (G2) can be subjected to a wash (defined in detail in the text which follows).

In a further preferred embodiment of the present invention, mixture (G1) is discharged as an output from the apparatus in which the chemical reaction is performed, conducted through a phase separation unit, especially into a phase separator, and then fed into the rectifying column (R1). In other words, this means that, after the performance of the chemical reaction, preferably the hydrocarbon conversion, especially the isomerization, and prior to performance of the inventive removal of mixture (G1b), especially of hydrogen halide, from the rectifying column (R1), an intermediate step is performed. In this intermediate step, the ionic liquid present in mixture (G1) is fully or at least partly removed from mixture (G1), and then mixture (G1) depleted of acidic ionic liquid is fed into the rectifying column (R1).

Preferably at least 90%, more preferably at least 99%, of the ionic liquid is removed from mixture (G1) in the phase separation unit and optionally recycled into the apparatus in which the chemical reaction is performed. Especially preferably, the ionic liquid removed from mixture (G1) in the phase separation unit is recycled fully or partly into the apparatus for performance of a chemical reaction, preferably for performance of a hydrocarbon conversion, especially for performance of an isomerization.

The above-described further preferred embodiment of the present invention is additionally illustrated in FIG. 2. In FIG. 2, the abbreviations, arrows and other symbols have similar meanings to those explained above for FIG. 1; PT means phase separation unit, IL means ionic liquid. For the sake of completeness, it is pointed out that the embodiment according to FIG. 2 can also be combined with the wash described below.

In a further preferred embodiment of the present invention, mixture (G2) discharged from the rectifying column (R1) is subjected to a wash according to step d) below. In the optional step d) of the process according to the invention, mixture (G2) is washed with an aqueous medium to obtain a mixture (G3) comprising at least one hydrocarbon and not more than 100 ppm by weight, preferably not more than 10 ppm by weight, of hydrogen halide (HX) (based on the total weight of (G3)).

In the context of the process according to the invention, it is preferable that mixture (G3) obtained in process step d) (wash step d), with regard to the composition and/or amount of the organic compounds present therein, especially of the hydrocarbons, corresponds completely or at least substantially to mixtures (G1) and (G2). The expression “corresponds substantially” for mixture (G3) comprehends the analogous statements to those made above in connection with mixtures (G1) and (G2). Especially preferably, mixture (G3) does not comprise any further components apart from at least one organic compound, preferably at least one hydrocarbon, and not more than 100 ppm by weight, preferably not more than 10 ppm by weight of hydrogen halide.

Preference is given to performing process step d) in such a way that the wash according to step d) comprises at least two wash steps:
d1) in a first wash step, the aqueous medium used has a pH >9, preferably >12,
d2) in a second wash step, the aqueous medium used has a pH between 5 and 9, preferably between 6 and 8.

The aqueous medium in the first wash step preferably comprises an alkali metal hydroxide, especially preferably NaOH. The aqueous medium in the second wash step is preferably water, especially preferably demineralized water.

Optionally, process step d) can be performed in such a way that step d2) can be performed prior to step d1). In this process variant, washing is thus effected first with an aqueous medium of relatively low pH, followed by the wash with an aqueous medium of higher pH. In addition, it is possible to perform several steps d1) and several steps d2) in succession, optionally in alternating sequence.

Preference is given in the context of the present invention to performing wash step d) in two stages, first step d1) and then step d2).

In one embodiment of the present invention, only a one-stage wash step d) is performed, in which case the aqueous medium has a pH of 5 to 9, preferably between 6 and 8, and is especially preferably demineralized water.

In addition, it is preferable in the context of the present invention that step d) is performed using at least one dispersion and phase separation unit or at least one extraction column per wash stage. The dispersion and phase separation unit is preferably a mixer-settler apparatus, a combination of static mixers with phase separators or a combination of mixing pump with phase separator.

It is additionally preferred in the context of the present invention that, in the case of a multistage, especially two-stage, wash, mixture (G2) is conducted in countercurrent to the aqueous medium. It is especially preferred in the context of the present invention that mixture (G2) discharged from the rectifying column (R1) is washed with the aqueous medium (according to step d)) without any intermediate steps.

In another embodiment, the wash step d) is performed in a multistage mixer-settler apparatus, preferably operated in countercurrent, or extraction is effected with water in an extraction column operated in countercurrent. In the case of the mixer-settler apparatus or extraction column, a further wash stage is preferably connected downstream thereof in flow direction of the mixture (G2) (comprising the organic compounds, especially the hydrocarbons), this being fed with fresh water. In the aqueous outlet thereof is an apparatus for continuous measurement of the pH or the electrical conductivity, in order thus to monitor the complete removal of the non-hydrocarbon components, especially HCl.

In the context of the present invention, preferably cyclohexane is isolated from mixture (G2) and/or mixture (G3). Preference is given to isolating cyclohexane from mixture (G3), i.e. after performance of the optional wash step. Processes and apparatuses for removal of cyclohexane from such mixtures are known to those skilled in the art.

### EXAMPLES

The present invention is to be illustrated hereinafter by examples.

For the simulation calculation, BASF's own software Chemasin was used (in the case of use of the commercially available software Aspen Plus (manufacturer: AspenTech, Burlington/Mass., USA), the same results would be obtained). The following substances or mixtures are used for the example calculation:

a) Hydrocarbon mixture (A) having the composition

| | |
|---|---|
| 2-methylpentane | 0.66% by wt. |
| 3-methylpentane | 1.96% by wt. |
| n-hexane | 28.85% by wt. |
| methylcyclopentane | 51.00% by wt. |
| cyclohexane | 17.45% by wt. |
| 2,3-dimethylbutane | 0.05% by wt. |
| other hydrocarbons | 0.03% by wt. |

b) Hydrogen chloride gas (B)
c) Ionic liquid (IL), specifically trimethylammonium heptachlorodialuminate (TMA-IL)

For the examples described, (A) and (B) are mixed such that the resulting mixture G1(-IL) after the phase separation has an HCl content of 1.5% by weight.

### 1. Example with HCl Removal or Recycling by Means of Rectification Column with Partial Condensation (R1+PK)

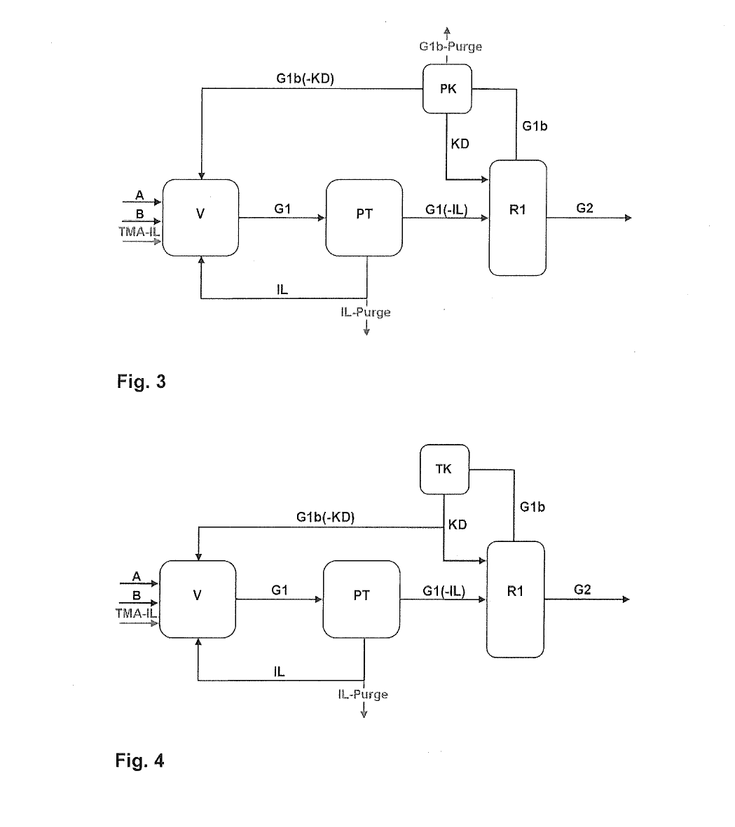

Example 1 is shown schematically in FIG. 3. In an apparatus V, an isomerization of a hydrocarbon mixture (A) takes place in the presence of a trimethylammonium heptachlorodialuminate compound (TMA-IL), which serves as a catalyst. The volume ratio of ionic liquid to organic phase is 5. In addition, hydrogen chloride gas (B) for stabilization of the IL and the recycle streams G1b(-KD) from a partial condenser PK and IL from a phase separation are fed in. For apparatus V, an operating pressure of 3.5 bar (abs) and a temperature of 50° C. are assumed. The resulting mixture G1 is passed into an apparatus for phase separation PT. In a simplification, it is assumed for this apparatus that the TMA-IL is removed and recycled completely and as a pure IL phase. The organic phase G1(-IL) is passed into the rectification column R1. For this column, three theoretical plates are assumed, the feed being effected to the middle plate, plate 2. At the top, the gaseous mixture G1b is drawn off and partly condensed in the partial condenser PK. The condensate is recycled to the upper plate, plate 1, of the column. The uncondensed gas stream G1b(-KD) is recycled into the apparatus V. The “purge” refers to the substreams of G1 and IL which can optionally be removed from the process. The liquid mixture G2 is discharged from the bottom of R1 and can be treated by washing in a downstream step (not part of this example calculation). The rectification column R1 is equipped with an integrated reboiler. It is specified that 95% of the vapors G1 obtained is condensed and recycled fully to the column (reflux ratio RV=19 g/g). In addition, 98% by weight of the hydrocarbons present in G1 is drawn off via the bottom stream G2.

The calculated properties and compositions of the streams are shown in table 1.

TABLE 1
Properties and composition of the streams from example 1

| Stream: | | | A | B | G1 | IL | G1(-IL) | G1b | KD | G1b(-KD) | G2 |
|---|---|---|---|---|---|---|---|---|---|---|---|
| from apparatus | | | | | V | PT | PT | R1 | PK | PK | R1 |
| from plate | | | | | | | | 3 | | | 1 |
| to apparatus | | | V | V | PT | V | R1 | PK | R1 | V | |
| to plate | | | | | | | 2 | | 3 | | |
| Phase | | | fluid | gas | fluid | fluid | fluid | gas | fluid | gas | fluid |
| | | | cur. | cur. | cur. | cur. | cur. | cur. | cur. | cur. | cur. |
| Properties | | unit | value | value | value | value | value | value | value | value | value |
| Temperature | | ° C. | 50.000 | 50.000 | 50.000 | 50.000 | 50.000 | 114.514 | 77.201 | 77.201 | 120.784 |
| Pressure | | bar | 3.500 | 3.500 | 3.500 | 3.500 | 3.500 | 3.500 | 3.500 | 3.500 | 3.500 |
| Enthalpy | | kW | 0.139 | 0.000 | 1.526 | 1.383 | 0.143 | 0.559 | 0.157 | 0.023 | 0.361 |
| Mean molar mass | | kg/kmol | 84.791 | 36.461 | 275.218 | 362.251 | 83.151 | 82.026 | 84.322 | 54.064 | 84.791 |
| Thermal conductivity | | W/(m*K) | 0.113 | 0.015 | 0.143 | 0.150 | 0.112 | 0.021 | 0.100 | 0.017 | 0.094 |
| Viscosity eta | | mPa*s | 0.349 | 0.016 | 6.080 | 21.012 | 0.394 | 0.009 | 0.247 | 0.012 | 0.214 |
| Surface tension | | N/m | 0.018 | | 0.014 | 0.013 | 0.018 | | 0.013 | | 0.011 |
| Specific heat | | kJ/kg/K | 2.113 | 0.799 | 2.009 | 2.000 | 2.097 | 1.863 | 2.327 | 1.345 | 2.449 |
| Density | | kg/m³ | 695.555 | 4.750 | 1269.102 | 1385.511 | 702.028 | 8.907 | 645.335 | 6.496 | 630.625 |
| Mass flow rate | | kg/h | 5.000 | 0.000 | 54.978 | 49.799 | 5.180 | 3.595 | 3.415 | 0.180 | 5.000 |
| | | | cur. | cur. | cur. | cur. | cur. | cur. | cur. | cur. | cur. |
| Concentrations | MolM. | unit | value | value | value | value | value | value | value | value | value |
| 2-M-PENTANE | 86.179 | g/g | 0.0066 | | 0.0045 | | 0.0473 | 0.1131 | 0.1148 | 0.0861 | 0.0460 |
| 3-M-PENTANE | 86.179 | g/g | 0.0196 | | 0.0039 | | 0.0416 | 0.0785 | 0.0798 | 0.0547 | 0.0412 |
| HEXANE | 86.179 | g/g | 0.2885 | | 0.0198 | | 0.2099 | 0.3029 | 0.3088 | 0.1844 | 0.2106 |
| M-CY-PENTANE | 84.163 | g/g | 0.5100 | | 0.0113 | | 0.1203 | 0.1189 | 0.1217 | 0.0638 | 0.1223 |
| CYCLOHEXANE | 84.163 | g/g | 0.1745 | | 0.0515 | | 0.5470 | 0.2963 | 0.3053 | 0.1264 | 0.5621 |
| 2,2-DM-BUTANE | 86.179 | g/g | 0.0000 | | 0.0004 | | 0.0041 | 0.0177 | 0.0177 | 0.0175 | 0.0037 |
| 2,3-DM-BUTANE | 86.179 | g/g | 0.0005 | | 0.0014 | | 0.0145 | 0.0424 | 0.0429 | 0.0347 | 0.0138 |
| TMA-IL | 362.251 | g/g | | | 0.9058 | 1.0000 | | | | | |
| HCL | 36.461 | g/g | | 1.0000 | 0.0014 | | 0.0150 | 0.0298 | 0.0086 | 0.4323 | 0.0000 |

### 2. Comparative Example with HCl Removal or Recycling by Means of Rectification Column with Total Condensation (R1+TK)

Comparative example 2 is shown schematically in FIG. 4. In an apparatus V, an isomerization of a hydrocarbon mixture (A) takes place in the presence of a trimethylammonium heptachlorodialuminate compound (TMA-IL) which serves as a catalyst. The volume ratio of ionic liquid to organic phase is 5. In addition, hydrogen chloride gas (B) is supplied for stabilization of the IL, as are the recycle streams G1b(-KD) from a total condenser TK and IL from a phase separation. For apparatus V, an operating pressure of 3.5 bar (abs) and a temperature of 50° C. are assumed. The resulting mixture G1 is passed into an apparatus for phase separation PT. In a simplification, it is assumed for this apparatus that the TMA-IL is removed and recycled fully and as a pure IL phase. The organic phase G1(-IL) is passed into the rectification column R1. For this column, three theoretical plates are assumed, the feed being effected to the middle plate, plate 2. At the top, the gaseous mixture G1b is drawn off and fully condensed in the total condenser TK. 95% by weight of the condensate is recycled to the upper plate, plate 1, of the column. The rest of the condensate G1b(-KD) is recycled in liquid form into the apparatus V. The liquid mixture G2 is discharged from the bottom of R1 and can be treated in a subsequent step by washing (not part of this example calculation). The rectification column R1 is equipped with an integrated reboiler. It is specified that 95% of the condensate obtained is recycled to the column (reflux ratio RV=19 g/g). In addition, 98% by weight of the hydrocarbons present in G1 is drawn off via the bottom stream G2.

The calculated properties and compositions of the streams are shown in table 2.

TABLE 2
Properties and composition of the streams from comparative example 2

| Stream: | | | A | B | G1 | IL | G1(-IL) | G1b | KD | G1b(-KD) | G2 |
|---|---|---|---|---|---|---|---|---|---|---|---|
| from apparatus | | | | | V | PT | PT | R1 | TK | TK | R1 |
| from plate | | | | | | | | 3 | | | 1 |
| to apparatus | | | V | V | PT | V | R1 | TK | R1 | V | |
| to plate | | | | | | | 2 | | 3 | | |
| Phase | | | fluid | gas | fluid | fluid | fluid | gas | fluid | fluid | fluid |
| | | | cur. | cur. | cur. | cur. | cur. | cur. | cur. | cur. | cur. |
| Properties | | unit | value | value | value | value | value | value | value | value | value |
| Temperature | | ° C. | 50.000 | 50.000 | 50.000 | 50.000 | 50.000 | 78.579 | −58.261 | −58.261 | 120.784 |
| Pressure | | bar | 3.500 | 3.500 | 3.500 | 3.500 | 3.500 | 3.500 | 3.500 | 3.500 | 3.500 |
| Enthalpy | | kW | 0.139 | 0.000 | 1.526 | 1.383 | 0.143 | 0.464 | −0.104 | −0.005 | 0.361 |
| Mean molar mass | | kg/kmol | 84.791 | 36.461 | 275.217 | 362.251 | 83.149 | 54.032 | 54.032 | 54.032 | 84.791 |
| Thermal conductivity | | W/(m*K) | 0.113 | 0.015 | 0.143 | 0.150 | 0.112 | 0.017 | 0.187 | 0.187 | 0.094 |
| Viscosity eta | | mPa*s | 0.349 | 0.016 | 6.082 | 21.012 | 0.394 | 0.012 | 0.430 | 0.430 | 0.214 |
| Surface tension | | N/m | 0.018 | | 0.014 | 0.013 | 0.018 | | 0.026 | 0.026 | 0.011 |
| Specific heat | | kJ/kg/K | 2.114 | 0.799 | 2.009 | 2.000 | 2.097 | 1.332 | 1.799 | 1.799 | 2.449 |
| Density | | kg/m³ | 695.536 | 4.750 | 1269.150 | 1385.511 | 702.174 | 6.467 | 886.794 | 886.794 | 630.609 |
| Mass flow rate | | kg/h | 5.000 | 0.000 | 54.980 | 49.800 | 5.180 | 3.595 | 3.415 | 0.180 | 5.000 |
| | | | cur. | cur. | cur. | cur. | cur. | cur. | cur. | cur. | cur. |
| Concentrations | MolM. | unit | value | value | value | value | value | value | value | value | value |
| 2-M-PENTANE | 86.179 | g/g | 0.0066 | | 0.0044 | | 0.0468 | 0.0709 | 0.0709 | 0.0709 | 0.0459 |
| 3-M-PENTANE | 86.179 | g/g | 0.0195 | | 0.0039 | | 0.0414 | 0.0482 | 0.0482 | 0.0482 | 0.0411 |
| HEXANE | 86.179 | g/g | 0.2887 | | 0.0198 | | 0.2099 | 0.1792 | 0.1792 | 0.1792 | 0.2110 |
| M-CY-PENTANE | 84.163 | g/g | 0.5099 | | 0.0114 | | 0.1205 | 0.0684 | 0.0684 | 0.0684 | 0.1224 |
| CYCLOHEXANE | 84.163 | g/g | 0.1744 | | 0.0516 | | 0.5480 | 0.1614 | 0.1614 | 0.1614 | 0.5619 |
| 2,2-DM-BUTANE | 86.179 | g/g | 0.0000 | | 0.0004 | | 0.0039 | 0.0121 | 0.0121 | 0.0121 | 0.0036 |
| 2,3-DM-BUTANE | 86.179 | g/g | 0.0005 | | 0.0013 | | 0.0142 | 0.0274 | 0.0274 | 0.0274 | 0.0137 |
| TMA-IL | 362.251 | g/g | | | 0.9058 | 1.0000 | | | | | |
| HCL | 36.461 | g/g | | 1.0000 | 0.0014 | | 0.0150 | 0.4323 | 0.4323 | 0.4323 | 0.0000 |

### 3. Results of the Simulation Calculation

The results show that the use of a partial condenser PK can achieve significant depletion of hydrogen chloride from the hydrocarbon mixture G1(-IL) given a moderate temperature difference between top and bottom of the rectifying column R1 used. The difference between vaporization and condensation temperature is 43.6 K. Compared to this, the temperature difference in comparative example 2, in the case of use of a total condenser, is 179.0 K. It should be emphasized here that a temperature of −58.2° C. is required for the total condensation of stream G1b at 3.5 bar (abs), which is well below the standard cooling water temperatures and thus necessitates a costly and inconvenient cooling unit.

In addition, the hydrogen halide, owing to the recycling, can be reused in the process to an extent of nearly 100%. In the case of full return of the gas stream G1b(-KD), there are additionally no accompanying losses of product of value.

1-21. (canceled)

22. A process for treating an output from a chemical reaction in the presence of an ionic liquid, the output comprising a mixture (G1) and mixture (G1) comprising at least one organic compound and at least one hydrogen halide (HX), which comprises feeding mixture (G1) into a rectifying column (R1) comprising a partial condenser and a reboiler, and removing from (R1) a mixture (G1b) comprising at least 50% by weight of the hydrogen halide (HX) present in mixture (G1), and a mixture (G2) comprising a reduced amount of hydrogen halide (HX) compared to mixture (G1).

23. The process according to claim 22, wherein the chemical reaction is performed in the presence of an acidic ionic liquid having the composition K1AlₙX₍₃ₙ₊₁₎ where K1 is a monovalent cation, X is halogen and 1<n<2.5.

24. The process according to claim 22, wherein the chemical reaction is a hydrocarbon conversion or the organic compound is a hydrocarbon.

25. The process according to claim 22, comprising the following steps:
a) drawing off the gaseous mixture (G1b) from the rectifying section of the rectifying column (R1), mixture (G1b) comprising at least 50% of the amount of the hydrogen halide (HX) present in mixture (G1),
b) partially condensing gaseous mixture (G1b) drawn off in step a) in the partial condenser of (R1) to obtain at most 98% by weight of the total amount of (G1b) as condensate (KD), and removing the uncondensed portion of the gaseous mixture (G1b) comprising 10 to 90% by weight of hydrogen halide (HX) from the partial condenser,
c) at least partially recycling the condensate (KD) obtained in step b) into the rectifying column (R1).

26. The process according to claim 22, wherein mixture (G1) is fed into the rectifying column (R1) at a point below the feed of the condensate (KD) to (R1) or above the column bottom of (R1).

27. The process according to claim 26, wherein the rectifying column (R1) has a stripping section and a rectifying section.

28. The process according to claim 27, wherein (R1) is operated in such a way that the temperature in the stripping section is regulated with the heat output introduced into the reboiler of (R1).

29. The process according to claim 27, wherein heat output is introduced into the reboiler in the form of steam or steam condensate.

30. The process according to claim 22, wherein the temperature in the partial condenser is regulated via the amount or temperature of the cooling medium or the liquid level in the partial condenser.

31. The process according to claim 24, wherein the hydrocarbon conversion is selected from an alkylation, a polymerization, a dimerization, an oligomerization, an acylation, a metathesis, a polymerization or copolymerization, an isomerization, a carbonylation or combinations thereof.

32. The process according to claim 31, wherein the hydrocarbon conversion is an isomerization of methyl cyclopentane (MCP) to cyclohexane.

33. The process according to claim 22, wherein the hydrogen halide (HX) is hydrogen chloride (HCl).

34. The process according to claim 22, wherein mixture (G1b) drawn off from the rectifying column (R1) is recycled fully or partly into the apparatus in which the chemical reaction.

35. The process according to claim 22, wherein the acidic ionic liquid comprises, as a cation, an at least partly alkylated ammonium ion or a heterocyclic cation or, as an anion, a chloroaluminate ion having the composition AlₙCl₍₃ₙ₊₁₎ where 1<n<2.5.

36. The process according to claim 22, wherein mixture (G2) is washed with an aqueous medium to obtain a mixture (G3) comprising at least one hydrocarbon and not more than 100 ppm by weight, preferably not more than 10 ppm by weight of hydrogen halide (HX).

37. The process according to claim 22, wherein mixture (G1) comprises a hydrocarbon mixture comprising at least 80% by weight (based on the total amount of hydrocarbons) of hydrocarbons having at least 5 carbon atoms, preferably having at least 6 carbon atoms.

38. The process according to claim 37, wherein the hydrocarbon mixture comprises cyclohexane or a mixture of cyclohexane and at least one further hydrocarbon selected from methylcyclopentane (MCP), n-hexane, isohexanes, n-heptane, isoheptanes, methylcyclohexane or dimethylcyclopentanes.

39. The process according to claim 22, wherein mixture (G2) removed from the rectifying column (R1) is liquid when it leaves (R1) and is at most 150 K, preferably at most 100 K, hotter than the gaseous mixture (G1b) when it leaves the rectifying section of (R1).

40. The process according to claim 22, wherein mixture (G1) additionally comprises between 10 and 99% by weight of the ionic liquid.

41. The process according to claim 40, wherein mixture (G1) is discharged as an output from the apparatus in which the chemical reaction is performed, conducted through a phase separation unit, especially into a phase separator, and then fed into the rectifying column (R1).

42. The process according to claim 41, wherein at least 90% by weight of the ionic liquid present in mixture (G1) is removed from mixture (G1) in the phase separation unit and optionally fully or partly returned to the apparatus in which the chemical reaction, preferably the hydrocarbon conversion, is performed.

43. The process according to claim 22, wherein cyclohexane is isolated from mixture (G2) or mixture (G3).

44. The process according to claim 25, wherein in step b) at most 95% by weight of the total amount of (G1b) is obtained as condensate (KD).
